(19) United States
(12) Patent Application Publication
(10) Pub. No.: US 2013/0041134 A1
(43) Pub. Date: Feb. 14, 2013

(54) METHODS FOR PREPARING POLYETHYLENE GLYCOL MALEIMIDE USING N-(2-HYDROXYETHYL) MALEIMIDE AS A STARTING MATERIAL

(71) Applicants: John R. Handley, Duluth, MN (US); Jonathon B. McKannan, Huntsville, AL (US)

(72) Inventors: John R. Handley, Duluth, MN (US); Jonathon B. McKannan, Huntsville, AL (US)

(73) Assignee: SANGART ,INC., San Diego, CA (US)

(21) Appl. No.: 13/505,444

(22) PCT Filed: Nov. 2, 2010
(86) PCT No.: PCT/US2010/055155

§ 371 Date: Jul. 10, 2012

Related U.S. Application Data
(60) Provisional application No. 61/258,461, filed on Nov. 5, 2009.

(51) Int. Cl. C07D 207/40; C07K 1/107

(52) U.S. Cl. 530/362; 548/547; 530/409; 530/385

(57) ABSTRACT

The present invention relates generally to methods for preparing polyethylene glycol maleimide (“PEG-Mal”). More specifically, the present invention relates to methods for synthesizing desired molecular weight PEG-Mal by direct ethoxylation of N-(2-hydroxyethyl)maleimide under specific reaction conditions.

### CROSS REFERENCE TO RELATED APPLICATIONS

This application is a United Stage national stage entry under 35 U.S.C.371 based on International Application No. PCT/US10/55155 filed Nov. 2, 2010, entitled “Methods for Preparing Polyethylene Glycol Maleimide using N-(2-Hydroxyethyl)Maleimide as a Starting Material,” which claims priority to U.S. Provisional Application Ser. No. 61/258,461, filed on Nov. 5, 2009, entitled “Methods for Preparing Polyethylene Glycol Maleimide using N-(2-Hydroxyethyl)Maleimide as a Starting Material,” the contents of which are incorporated herein by reference as if set forth verbatim.

### TECHNICAL FIELD

The present invention relates generally to methods for preparing polyethylene glycol maleimide (“PEG-Mal”). More specifically, the present invention relates to methods for synthesizing desired molecular weight PEG-Mal by direct ethoxylation of N-(2-hydroxyethyl)maleimide under specific reaction conditions.

### BACKGROUND OF THE INVENTION

Modified hemoglobins are currently being developed as blood substitutes and oxygen carriers. In previous studies, it was observed that the molecular size of the modified hemoglobin had to be large enough to avoid being cleared by the kidneys and to achieve the desired circulation half-life. Blumenstein, et al., determined that this could be achieved at, or above, a molecular weight of 84,000 Daltons (“Da”) (“Blood Substitutes and Plasma Expanders,” Alan R. Liss, editors, New York, N.Y., pages 205-212 (1978)). In that study, the authors conjugated dextran of varying molecular weight to hemoglobin. They reported that a conjugate of hemoglobin (with a molecular weight of 64,000) and dextran (having a molecular weight of 20,000) “was cleared slowly from the circulation and negligibly through the kidneys.” Further, they found that increasing the molecular weight above 84,000 did not alter the clearance curves.

One way of achieving high molecular weight modified hemoglobin is to conjugate it to a synthetic polymer, such as polyalkylene oxide (“PAO”). Examples of PAO include, for example, polyethylene oxide (—(CH₂CH₂O)ₙ—), polypropylene oxide (—(CH(CH₃)CH₂O)ₙ—) or a polyethylene/polypropylene oxide copolymer (—(CH₂CH₂O)ₙ(CH(CH₃)CH₂O)ₙ—).

The most common polyalkylene oxide presently used to modify the surface of hemoglobin is polyethylene glycol (“PEG”) because of its pharmaceutical acceptability and commercial availability. In addition, PEG is available in a variety of molecular weights based on the number of repeating subunits of ethylene oxide (i.e. —OCH₂CH₂—) within the molecule. PEG formulations are usually followed by a number that corresponds to their average molecular weight. For example, PEG-200 has an average molecular weight of 200 Da and may have a molecular weight range of 190-210 Da.

In order to bond polyalkylene oxides to hemoglobin, the terminal end-groups of the polymer must first be converted into reactive functional groups. This process is frequently referred to as “activation”, and the product is called an “activated polyalkylene oxide.” In the past, PEG-OH was used to prepare PEG-halide, mesylate or tosylate, which was then converted to PEG-amine by performing a nucleophilic displacement reaction with aqueous ammonia (Hoffmann Reaction), sodium azide or potassium phthalimide (Gabriel Reagent). The reaction of PEG-halide with ammonia forms PEG-amine (“PEG-NH₂”) directly (See Zalipsky et al. Eur. Polym. J. 1983, 19:1177-)183), which could then be used “as is” for conjugation to —COOH groups found on some biologically active compounds.

More recently, PEG-NH, is used as an intermediate and can be further functionalized to bind groups other than -COOH. For example, PEG-NH, can be modified to contain a sulfhydryl activated group such as maleimide. In a reaction disclosed in U.S. Pat. No. 6,828,401, mPEG-maleimide (i.e. methoxy-PEG, or mPEG, to which a maleimide has been added) is prepared by reacting mPEG-OH with p-toluenesulfonyl chloride (a tosylating agent) and triethyleneamine (“TEA”, a base catalyst), in the presence of dichloromethane (an organic solvent) to produce mPEG-tosylate. This compound is then reacted with 28% ammonia water, which is then reacted with maleic acid anhydride in an organic solvent mixture of N,N-dimethylacetamide (“DMAC”) and N-cyclohexylpyrrolidinone (“CHP”) to produce a maleamic acid compound. This compound is then reacted with pentafluorophenyl trifluoroacetate in the presence of dichloromethane, or base catalyst such as diethylaniline (“DEA”) or diisopropylethylamine (“DIEA”), in an organic solvent mixture of dichloromethane and dimethyl formamide (“DMF”), to produce the mPEG-maleimide. However, this multi-step, multi-reagent method is cumbersome and time consuming.

In addition, mPEG is often inherently contaminated with high molecular weight bifunctional PEG (i.e. “PEG diol”) resulting from the production process. The amount of contaminant can range as high as 10 to 15% (Dust et al. 1990, Macromolecule 23:3742-3746). The contamination problem is further aggravated as the molecular weight of PEG increases. The purity of mPEG is especially critical for the production of PEGylated biotherapeutics, because the FDA will require a high level of reproducibility in the production processes and quality of the final product.

It has been reported that PEG-Mal can be produced from N-(2-hydroxyethyl)maleimide and ethylene oxide in the presence of a double metal cyanide catalyst (Thompson, M. S. “Synthesis toward the Production of Biocompatible Magnetic Nanoparticles with Tailored Surface Properties,” Dissertation dated Jul. 10, 2007, Virginia Polytechnic Institute). However, as reported by Thompson, this approach produces a product with a broad molecular weight distribution (3.3 PDI), which is highly undesirable in the practice of the present invention.

Accordingly, there is a need for a method of preparing PEG-Mal of a desired molecular weight that reduces the overall cost of preparation by limiting the number of reaction steps, reducing the amount of time to perform the reactions, minimizing contaminants and eliminating high dilution steps for product isolation and purification.

### SUMMARY OF THE INVENTION

The present invention relates generally to methods for preparing polyethylene glycol maleimide (“PEG-Mal”). More specifically, the present invention relates to methods for synthesizing desired molecular weight PEG-Mal by direct ethoxylation of N-(2-hydroxyethyl)maleimide under specific reaction conditions.

In one embodiment, the present invention pertains to a method for preparing PEG-Mal by first mixing N-(2-hydroxyethyl)maleimide with a polymerization catalyst to form a mixture, wherein the catalyst is not a double metal cyanide catalyst, and thereafter adding ethylene oxide to the mixture to produce PEG-Mal. These two steps are conducted in the presence of 100 parts per million or less of water and result in a PEG-Mal product with a polydispersity of less than 1.5.

In another embodment, the method of the present invention includes the additional step of reacting the PEG-Mal with a methylating reagent to form methoxy-PEG-Mal (mPEG-Mal). The methylating reagent can be, for example, dimethyl sulfate, diazomethane, methyl halide, methyl alcohol, triethylorthoformate, or dimethylcarbonate.

The catalyst can be either an anionic or cationic catalyst, such as NaOH, KOH, NaOCH₃, SbCl₅, SnCl₄, NaH, Ag₂O, BaO, NaNH₂ and NaO₂CCH₃.

The average molecular weight of the resultant PEG-Mal can vary, such as between 200 Da to 20,000 Da, between 4,000 Da and 6,000 Da, or between 4500 Da to 5000 Da.

The mPEG-Mal can also be used to conjugate mPEG to a protein by mixing it with a protein having (or modified to have) one or more amine or sulfhydryl groups. Such protein may, for example, be hemoglobin or albumin. Proteins can be modified to have more sulfhydryl groups by using a thiolating reagent, such as 2-iminothiolane, prior to mixing with mPEG-Mal.

Other aspects of the invention can be found throughout the specification.

### BRIEF DESCRIPTION OF THE DRAWINGS

FIG. 1 depicts a flow chart of an exemplary method for preparing mPEG-Mal by ethoxylation of N-(2-hydroxyethyl)maleimide.

### DETAILED DESCRIPTION OF THE INVENTION

The present invention relates generally to methods for preparing polyethylene glycol maleimide (“PEG-Mal”). More specifically, the present invention relates to methods for synthesizing desired molecular weight PEG-Mal by direct ethoxylation of N-(2-hydroxyethyl)maleimide under specific reaction conditions.

In the description that follows, a number of terms used in the field of molecular biology, immunology and medicine are extensively utilized. In order to provide a clear and consistent understanding of the specification and claims, including the scope to be given such terms, the following non-limiting definitions are provided.

When the terms “one,” “a,” or “an” are used in this disclosure, they mean “at least one” or “one or more,” unless otherwise indicated.

The term “activated polyethylene glycol” or “activated PEG” as used herein refers to a PEG molecule that has at least one functional group. A functional group is a reactive moiety that interacts with, for example free amines, sulfhydryls or carboxyl groups on a molecule to be conjugated with PEG. For example, one such functional group that interacts with free sulfhydryls in a molecule would be a maleimide group. Correspondingly, a functional group that interacts with a free amine on a molecule would be a succinimide group.

The term “catalyst” as used herein refers to a compound, or element, that facilitates the reaction of interest by lowering the rate-limiting free energy of the transition state of the reaction resulting in a larger reaction rate at the same temperature. However, unlike other reagents of the reaction, catalysts are not consumed by the reaction.

The term “hemoglobin” (“Hb”) as used herein refers generally to the protein contained within red blood cells that transports oxygen. Each molecule of hemoglobin has 4 subunits, 2 alpha chain subunits and 2 beta chain subunits, which are arranged in a tetrameric structure. Each subunit also contains one heme group, which is the iron-containing center that binds oxygen. Thus, each hemoglobin molecule can bind 4 molecules of oxygen.

The term “MalPEG-Hb” as used herein refers to Hb to which PEG-Mal has been conjugated. The conjugation is performed by reacting PEG-Mal (e.g., MW_{PEG}=5,000) with surface amine or thiol groups of the Hb to form MalPEG-Hb. Surface thiol groups can be found in native cysteine residues present in the amino acid sequence of the protein, or free amine groups in the amino acid sequence can be modified to contain a thiol group.

The term “alkylation” as used herein refers to the modification of a compound by the addition of an alkyl group, which is also referred to as “methylation” when the alkyl is a methyl group. For example, the terminal hydroxyl group of polyethylene glycol can be methylated by the replacement of the hydroxyl hydrogen with a methyl group, also known as mPEG, with the “m” referring to the added methyl group.

The term “mixture” or “mixing” as used herein refers to a mingling together of two or more substances without the occurrence of a reaction by which they would lose their individual properties. The term “solution” refers to a liquid mixture and the term “aqueous solution” refers to a solution that contains some water and may also contain one, or more, other liquid substances with water to form a multi-component solution.

The term “modified Hb” as used herein refers to, but is not limited to, Hb altered by a chemical reaction such as intra- and inter-molecular cross-linking, genetic manipulation, polymerization, and/or conjugation to other chemical groups (e.g., PAOs, for example PEG, or other adducts such as proteins, peptides, carbohydrates, synthetic polymers and the like). In essence, Hb is “modified” if any of its structural, or functional, properties have been altered from its native state. As used herein, the term “hemoglobin” or “Hb” by itself refers both to native (unmodified) Hb as well as modified Hb.

The term “oxygen affinity” as used herein refers to the avidity with which an oxygen carrier, such as Hb, binds molecular oxygen. This characteristic is defined by the oxygen equilibrium curve, which relates the degree of saturation of Hb molecules with oxygen (Y axis) to the partial pressure of oxygen (X axis). The position of this curve establishes the value, P50, i.e., the partial pressure of oxygen at which the oxygen carrier is half-saturated with oxygen, and is thus inversely related to oxygen affinity. Hence, the lower the P50, the higher the oxygen affinity. The oxygen affinity of any oxygen carrying substance such as whole blood (and components of whole blood, such as red blood cells and hemoglobin) or modified Hb can be measured by a variety of methods known in the art. (see, e.g., Winslow et al., J. Biol. Chem. 1977, 252(7):2331-2337). Oxygen affinity may also be determined using a commercially available HEMOX^{TM} Analyzer (TCS Scientific Corporation, New Hope, Pa.). (see, e.g., Vandegriff and Shrager in “Methods in Enzymology” (Everse et al., eds.) 232:460 (1994)).

The term “polyethylene glycol” or “PEG” as used herein refers to a-Hydro-ω-hydroxypoly-(oxy-1,2-ethanediyl) in the form of a liquid or solid, of the general chemical formula H(OCH₂CH₂)ₙOH, where n is greater than, or equal to, 4. Any PEG formulation, substituted or unsubstituted, is encompassed by this term. PEGs are commercially available in a number of formulations (e.g., Carbowax™ (Dow Chemical, Midland, Mich.), Poly-G® (Arch Chemicals, Norwalk Conn.), and Solbase).

The term “polyethylene glycol conjugated hemoglobin” or “PEG-Hb” as used herein refers to Hb that has been covalently conjugated with PEG.

The term “surface-modified hemoglobin” as used herein refers to Hb described above to which chemical groups have been attached to surface exposed amino acid residues. Such chemical groups include, for example, polymers such as dextran or PAO. These groups are usually attached covalently. The term “surface modified oxygenated Hb” refers to Hb that is in the “R” state when it is surface modified.

The term “thiolation” as used herein refers to the addition of sulfhydryl groups onto a molecule for binding thiol reactive compounds. For example, the number of sulfhydryl groups on Hb may be increased by reacting free amines present in the protein with a thiolating reagent, such as iminothiolane, thereby converting the free amine to a free sulfhydryl. These free sulfhydryls are then available for reaction with a thiol reactive moiety, such as the maleimide of PEG-Mal.

### Functional Groups

A number of molecules containing functional groups are available commercially to permit the modification of proteins by the addition of other molecules. These molecules, such as polyethylene glycol, are usually activated at their termini by adding one or more functional groups thereto. Two common functional groups utilized for this purpose are succinimides and maleimides. A succinimide is a cyclic imide with the formula C₄H₅NO₂, which is reactive with the free amines in lysine and histidine within the sequence of the protein. In comparison, a maleimide is a cyclic unsaturated imide of the formula H₂C₂(CO)₂NH, which is reactive with the free sulfhydryls in cysteine residues and can also react with free amines in lysine and histidine residues to a lesser extent. The succinimides form a stable leaving group from an active ester of carbonate, whereas the maleimides react directly with a sulfhydryl group to form a covalent bond.

According to the present invention, a maleimide functional group is not added to an existing polymer. Instead, N-(2-hydroxyethyl)maleimide is used as a starting material, which already includes the maleimide functional group.

### Polymerization by Ethoxylation

Polyethylene glycol is usually produced by the polymerization of ethylene oxide in water. Ethylene glycol and its oligomers are exemplary starting materials. Polymer chain length depends on the ratio of reactants, length of reaction and reaction temperature. When the starting material is ethylene glycol, this reaction is represented by the chemical equation:

HOCH₂CH₂OH+ₙ(CH₂CH₂O)→HO(CH₂CH₂O)ₙ₊₁H

Depending on the type of catalyst, the mechanism of polymerization can be cationic or anionic.

When polymerization is conducted via anionic mechanisms, the catalyst works by removing “active” hydrogen from the starting material to be ethoxylated. “Active” hydrogen is hydrogen connected to a hetero-atom (oxygen, nitrogen, etc.) that can be removed to form a reactive anion. For instance, the hydrogen in the hydroxyl group of ethylene glycol is the active hydrogen in the above described reaction. Hydrogen ion removal is accomplished by stripping water from the starting material in the presence of a catalyst prior to the addition of ethylene oxide. When added, the ethylene oxide reacts with the anion to form an ethoxylate anion. In turn, the ethoxylate anion can then react with an ethylene oxide, adding another ethylene oxide unit to continue the polymerization process. Once the desired reaction product is achieved, acid is added to the mixture to protonate the anionic form of the ethoxylate.

In the practice of the present invention, the starting material is N-(2-hydroxyethyl)maleimide. Accordingly, polymerization by ethoxylation as described above results in formation of an activated polyethylene glycol polymer, with a malemide functional group at one terminus.

### Catalysts

The present invention utilizes a catalyst capable of catalyzing polymerization via either a cationic or anionic mechanism to achieve a final product with a desirable polydispersity index (“PDI”), which is a measure of the molecular weight distribution of a polymeric compound. PDI is a unitless number defined as the ratio between weight average molecular weight and number average molecular weight (PDI=M_{w}/Mₙ). As the polymer chains in a particular polymer approach a uniform chain length, the PDI approaches 1. For a pharmaceutical applications in particular, a PDI near 1 is desired. According to the present invention the PDI is generally less than 1.5, typically less than 1.2 and most typically less than 1.05.

The present invention cannot be practiced using double metal cyanide (DMC) catalysts, because such catalysts are inefficient when using low molecular weight starting compounds, and result in the production of polymeric products from such starting materials that have a high level of polydispersity (>1.5 PDI). Accordingly, in the practice of the present invention which utilizes N-(2-hydroxyethyl)maleimide as a starting material, such catalysts would not be suitable.

Accordingly, in one embodiment, alkali catalysts, such as sodium hydroxide (NaOH), potassium hydroxide (KOH) and sodium carbonate (Na₂CO₃) may be used. In another embodiment, alkali earth catalysts (i.e., alkali transition metal oxides) are also suitable. These catalysts function by removing the active hydrogen from the N-(2-hydroxyethyl)maleimide starting material to form an anionic intermediate.

The ethoxylation process of the present invention may also be catalyzed with the use of Lewis acids (for example, boron trifluoride (BF₃), antimony pentachloride (SbCl₅), tin (IV) chloride (SnCl₄), or aluminum alkylates) or Bronsted acids (for example, p-toluene sulphonic acid, fluorosulfonic acid or perchloric acid). Acidic catalysts complex with the oxygen atom in ethylene oxide to render it susceptible to attack by the active oxygen in the starting material.

Narrow range ethoxylation catalysts (“NREs”) are also useful in the practice of the present invention. Useful examples of NRE catalysts are taught in U.S. Pat. Nos. 4,967, 016; 5,162,589; and 5,844, 115.

In one embodiment, a stoichiometric amount of catalyst is added to the N-(2-hydroxyethyl)maleimide to reduce the amount of residual catalyst, such as sodium methoxide (Na)CH₃), that would react directly with ethylene oxide to form mPEG-OH.

### Solvents

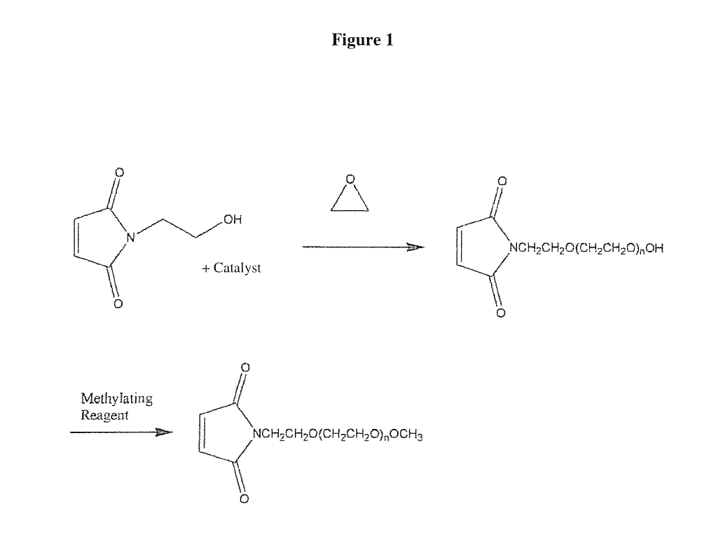

The present invention provides methods for the preparation of activated Mal-PEG from N-(2-hydroxyethyl)maleimide (see FIG. 1). This reaction is customarily carried out in the presence of a solvent, which offers the benefit of better reaction control, increased ease of agitation and improved reactant distribution within the reactor.

The choice of solvent for this reaction should be from among those that are capable of dissolving or maintaining the solubility of the catalyst, starting material, ethylene oxide and polymerization product, from which water can easily be removed and that are not reactive with the reactants or products. Typical solvents include, but are not limited to, aromatic hydrocarbon solvents, such as toluene, xylene, benzene, isopropyl benzene, and others in this class, ethereal solvents, such as dimethoxy ethanol, dimethyldiethyleneglcyol, tetrahudrofuran and others representative of this class and halogenated aromatic hydrocarbons, halocarbons and hydrocarbons.

In the typical anionic ethylene oxide polymerization of a substituted polyethylene glycol compound, such as that disclosed in U.S. Pat. No. 7,199,193, the rate of initiation is slower than the rate of propagation and the molecular weight distribution range is expected to broaden. A polyether aprotic solvent, however, has the ability to complex with the metallic component of an anionic catalyst, thereby making the anionic portion of the catalyst more available, increasing the rate of initiation with respect to the rate of propagation, thus promoting a narrower molecular weight distribution of the substituted PEG.

### Polymerization Conditions

Since water initiates ethylene oxide polymerization to form PEG-diol, and because PEG-diol is an undesired byproduct in the formation of PEG-Mal according to the instant invention, it is beneficial to minimize the water concentration during the reaction. In one embodiment, the water concentration should be less than 100 ppm. Ideally, it should be less than 50 ppm, or less than 10 ppm. Sources of water include water inherently present in the solvent, the starting material, the ethylene oxide, and water generated by dehydration of a polyethylene glycol alcohol.

Ethylene oxide is commercially available having a water content of less than 5 parts per million (“ppm”) by weight. Any nitrogen directed to the reactor is dried to a dew point of less than −100° C., using for example, a Drierite gas drying system (W. A. Hammond Drierite Company, Xenia, Ohio). Additions to the reactor should be made in a manner that excludes contamination by atmospheric moisture. The reactor system is best dried by carrying out a reaction and discarding the first batch. The system is then rinsed with dry solvent to remove the reaction product of the first batch and sealed in preparation for the next batch.

The temperature of the reaction will vary depending on the reactants and will range from about 80° to about 140° C. Reactor pressure is chosen to suit the pressure rating of the reactor, but is generally from about 15 to less than 100 pounds per square inch absolute (“psia”). The molar ratios of reactants are optimized to produce a polymer of the desired molecular weight and polydispersity (i.e., having a PDI less than 1.5).

### Protein Modification

A variety of proteins may be modified, or surface decorated, with PEG-Mal prepared by the methods of the present invention. The maleimide of PEG-Mal interacts predominantly with the free sulfhydryls of cysteine present in a protein, but is also known to react with free amines of lysine and histidine amino acid in the protein sequence to a lesser degree. Whether a free sulfhydryl, or amine, is available for binding will depend on the structure of the protein. For example, Hb has a fixed number of amino acid residues that may be accessed for conjugation to PEG-Mal as shown in the chart below.

| Residues | Positions |
|---|---|
| Alpha chain | |
| Lys | 7, 11, 16, 40, 56, 60, 61, |
| | 90, 99, 127, and 139 |
| Cys | 104 |
| His | 20, 45, 50, 58, 72, 87, |
| | 112 and 122 |
| Val | 1 |
| Beta chain | |
| Lys | 8, 17, 59, 61, 65, 66, 82, |
| | 95, 120, 132 and 144 |
| Cys | 93 and 112 |
| His | 2, 63, 77, 92, 97, 116, |
| | 117, 143 and 146 |
| Val | 1 |

However, their ability to bind PEG-Mal depends on their availability, which depends on the structural conformation of the Hb during the conjugation reaction and their reactivity. Because Hb changes upon binding oxygen, at least two structural conformations of the protein are available during conjugation. These structural conformations regulate which amino acid residues are available for conjugation. The chart below indicates the different amino acid residues available for conjugation based on whether the Hb is oxygenated or deoxygenated during the conjugation reaction.

Conjugation sites available

| | |
|---|---|
| Oxygenated Hb | Deoxygenated Hb |

Alpha

| | |
|---|---|
| Lys 7 | Lys 7 |
| — | Lys 11 |
| Lys 16 | Lys 16 |
| Lys 40 | — |
| — | Lys 139 |
| — | His 20 |
| — | Val 1 |

Beta

| | |
|---|---|
| Lys 8 | Lys 8 |
| Lys 17 | Lys 17 |
| Lys 59 | Lys 59 |
| — | Lys 61 |
| Lys 66 | Lys 66 |
| Lys 132 | Lys 132 |
| — | His 77 |
| — | His 97 |
| — | His 146 |
| Cys 93 | Cys 93 |

In one embodiment of the present method, the surface modification of the Hb takes place when the protein is in a deoxygenated, or R, state.

Hb is merely a representative protein and only one of a vast number of available proteins derived from humans or non-human animals that may be surface-modified with the PEG-Mal of the present invention. Consequently, the protein, and more particularly the Hb selected for surface modification, is not limited by its source. In this case, the Hb may be either native (unmodified), chemically modified, such as intra- or intermolecular cross-linked, or recombinantly engineered. Human α- and β-globin genes have both been cloned and sequenced (Liebhaber, et al., PNAS 1980, 77: 7054-7058; Marotta, et al., J. Biol. Chem. 1977, 353: 5040-5053 ((-globin cDNA)). In addition, many recombinantly produced modified Hbs have now been produced using site-directed mutagenesis, although these “mutant” Hb varieties were reported to have undesirably high oxygen affinities (e.g., Nagai, et al., PNAS 1985, 82:7252-7255). In one embodiment, the Hb of the present invention is stroma free and endotoxin free.

In certain proteins, conformation may be critical for desired, or proper, function. In the case of Hb, there are two distinct conformations, the “T” or oxygenated state and “R” the deoxygenated state. Conjugation of Hb to PAOs has been performed in both these states. U.S. Pat. No. 6,844,317 describes conjugating Hb in the oxygenated or “R” state to enhance the oxygen affinity of the resultant Hb. This is accomplished by equilibrating the Hb with the atmosphere prior to conjugation. Others describe a deoxygenation step prior to conjugation to diminish the oxygen affinity and increase structural stability to withstand the physical stresses of chemical modification, diafiltration and/or sterile filtration and sterilization (see U.S. Pat. No. 5,234,903).

### Thiolation

The use of succinimidyl functional groups for surface modification of proteins is well known and utilized regularly for preparation of reagents for diagnostic assays. However, when a protein so modified is introduced into a living system, these bonds are easily destroyed. This was observed when PAOs containing a succinimidyl functional group were utilized for binding free ε-amines available on the surface of Hb (Larwood and Szoka, 1984, J. Labeled Compounds Radiopharm. 21:603-14). When introduced into the blood stream of an animal, the ester bond formed between the polyalkylene chain and the succinimidyl group was easily hydrolyzed. To address this issue, PAOs were activated to produce urethane linkages with ε-amino groups of Hb, which are less susceptible to hydrolytic degradation (U.S. Pat. No. 5,234,903). Other methods have been utilized that employ thiolation of the ε-amines of Hb for binding PAOs having maleimide functional groups (U.S. Pat. No. 6,844,317). The thioester bonds formed under these methods are less susceptible to degradation (U.S. Patent Application No. 2006/0135753).

A variety of methods are known in the art for thiolation of proteins, such as Hb. These include, for example, thiolating amines by reaction with succinimidyl 3-(2-pyridyldithio)propionate followed by reduction of the 3-(2-pyridyldithio)propionyl conjugate with dithiothreitol (“DTT”), or tris(2-carboxyethyl)phosphine (“TCEP”). This reaction releases the 2-pyridinethione chromophore, which can be used to determine the degree of thiolation. Amines can also be indirectly thiolated by reaction with succinimidyl acetylthioacetate, followed by removal of the acetyl group with 50 mM hydroxylamine, or hydrazine, at near-neutral pH. In addition, 2-iminothiolane can be used to convert free amine groups on a protein into active thiol groups.

Accordingly, in one embodiment of the present invention, the Hb is thiolated prior to conjugation with PEG-Mal to increase the number of thiol groups available for conjugation.

### Methylation

The term “methylation” refers to an alkylation process wherein a methyl (—CH₃) group is delivered to a molecule. This is commonly performed using electrophilic methyl sources, such as iodomethane, dimethyl sulfate or dimethyl carbonate. More powerful methylating reagents, such as methyl triflate or methyl fluorosulfonate, are less commonly used. All of these reagents react via S_{N}2 nucleophilic substitution. One such reaction, wherein the hydroxyl group of an alcohol (“R—OH”) is methylated to give a methylated ether (“R—OCH₃”), is represented by the chemical reaction:

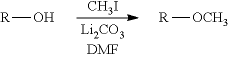

In one embodiment of the present invention, the PEG-Mal is methylated by converting the hydroxyl group to a methyl group as described above. Since the methyl group is relatively nonreactive, it results in formation of a monofunctional PEG intermediate that, when conjugated to Hb or any other protein, will not result in the formation of intermolecularly crosslinked protein dimers or multimers.

### EXAMPLES

### Example 1

### Preparation of N-poly(ethyleneglycol)maleimide (PEG-Mal)

Step 1: Clean and prepare a suitable 500 gallon reactor vessel for use. Carefully dry the vessel interior by purging with high pressure steam then rinsing with toluene and purging with a stream of dry nitrogen. Place 50 kilograms (“Kg”) dry toluene, 18.5 Kg (0.131 KMole) N-(2-hydroxyethyl)maleimide, 7.02 Kg (0.131 KMole) sodium methoxide and 106.9 Kg methanol in the reactor. Heat the reactor to 100-110° C. and remove methanol by distillation.

Step 2: Begin adding ethylene oxide (“EO”) to the reactor at a suitable rate. Add approximately 75 Kg of EO while maintaining the temperature in the desired range. Then cool the reactor mixture to ˜50° C. and remove approximately 70% of the total weight of material in the reactor and discard. This step is done to minimize diol formation resulting from the presence of water contamination in the initial reaction mixture.

Step 3: Raise the temperature of the reaction mixture to 100-110° C. and begin additional EO and a rate of 3-5 Kg/hour while maintaining the temperature below 120° C. When the required quantity of EU (approximately 170 Kg) has been added to the reactor, allow the mixture to cool to ˜80° C. Add sufficient acetic acid to the reactor to precisely neutralize any alkaline substances that are present in the mixture. At 80° C., purge the reaction mixture with nitrogen to remove any residual EO and any remaining volatile compounds such as toluene and acetic acid. Transfer the product through a filter (if possible, to remove sodium acetate) to a flaker and package.

### Example 2

### Preparation of Methoxy poly(ethyleneglycol)-N-methoxy-maleimide (mPEG-Mal)

Approximately 3.12 grams (0.22 moles, 1.1 equivalent (“eq”)) of methyl iodide was added to a solution of 100 grams (0.02 moles, 1.0 eq) of N-poly(ethyleneglycol)maleimide in 700 ml of tetrahydrofuran (very low water content) while stirring at room temperature and under a nitrogen atmosphere. Then, 0.51 grams (0.021 moles, 1.05 eq) of sodium hydride was added over a period of approximately one-half hour. Care was taken to ensure that no moisture contacted either the reaction mixture or the sodium hydride. Following the addition of the sodium hydride, the reaction mixture was stirred for an additional two hours.

The reaction mixture was distilled at reduced pressure until all traces of solvent were removed. The resulting residue was dissolved in approximately 100 mL of dichloromethane and stirred until solution was complete. This solution was then added dropwise to a flask containing 3 L of isopropanol (very low water content) at approximately 20° C. After agitating the resultant slurry for one-half hour, the mixture was filtered, washed with 300 ml of isopropanol and dried under vacuum to yield ˜95 grams of product.

The examples set forth above are provided to give those of ordinary skill in the art a complete disclosure and description of how to make and use the subject invention, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed:

1. A method for preparing polyethylene glycol maleimide (PEG-Mal) comprising the steps of:
a) mixing N-(2-hydroxyethyl)maleimide with a polymerization catalyst to form a mixture, wherein the catalyst is not a double metal cyanide catalyst; and
b) adding ethylene oxide to the mixture to produce PEG-Mal;
wherein steps a) and b) are conducted in the presence of 100 parts per million or less of water; and
wherein the PEG-Mal has a polydispersity index of less than 1.5.

2. The method according to claim 1 further comprising the step of:
c) reacting the PEG-Mal with a methylating reagent to form methoxy-PEG-Mal (mPEG-Mal).

3. The method according to claim 1, wherein the methylating reagent is selected from the group consisting of dimethyl sulfate, diazomethane, methyl halide, methyl alcohol, triethylorthoformate, and dimethylcarbonate.

4. The method according to claim 1, wherein the catalyst is selected from the group consisting of NaOH, KOH, NaOCH₃, SbCl₅, SnCl₄, NaH, Ag₂O, BaO, NaNH, and C₂H₃NaO₂.

5. The method according to claim 1, wherein the PEG-Mal has an average molecular weight between 200 Da to 20,000 Da.

6. The method according to claim 1, wherein the PEG-Mal has an average molecular weight between 4,000 Da and 6,000 Da.

7. The method according to claim 1, wherein the PEG-Mal has an average molecular weight of 4500 Da and 5500 Da.

8. A method for preparing a methoxy-PEG (mPEG) conjugated protein comprising the steps of:
mixing mPEG-Mal according to claim 2 with a protein having one or more amine or sulfhydryl groups.

9. The method according to claim 8, wherein the protein is hemoglobin or albumin.

10. The method according to claim 9, wherein the hemoglobin is modified with a thiolating reagent prior to mixing with mPEG-Mal.

11. The method according to claim 10, wherein the thiolating reagent is 2-iminothiolane.
